(19) United States
(12) Patent Application Publication
(10) Pub. No.: US 2018/0196926 A1
(43) Pub. Date: Jul. 12, 2018

(54) SYSTEM AND METHOD FOR GENERATING ANTIBODY LIBRARIES

(71) Applicant: IGC BIO, INC., Brookline, MA (US)

(72) Inventors: Lior ZIMMERMAN, Tel Aviv (IL); Dror Baran, Tel Aviv (IL)

(73) Assignee: IGC BIO, INC., Brookline, MA (US)

(21) Appl. No.: 15/863,927

(22) Filed: Jan. 7, 2018

Related U.S. Application Data
(60) Provisional application No. 62/443,172, filed on Jan. 6, 2017.

(51) Int. Cl. G06F 19/00; C07K 16/18; C40B 50/06; C40B 50/02; G06F 19/22; B01J 19/00

(52) U.S. Cl. CPC G06F 19/702; C07K 16/18; C40B 50/06; C40B 50/02; G01N 2500/04; G06F 19/701; B01J 19/0046; C07K 2317/10; G06F 19/22

(57) ABSTRACT

The invention relates to system and method for generating an antibody library. Specifically, the invention relates to a computer-implemented system and method for generating a library of antibodies based on a predetermined epitope.

### FIELD OF THE INVENTION

The invention relates to system and method for generating an antibody library. Specifically, the invention relates to a computer-implemented system and method for generating a library of antibodies based on a predetermined epitope.

### BACKGROUND OF THE INVENTION

Monoclonal antibodies have been functioning as therapeutic, diagnostic and research agents since the 1970s. One of the major advancements of the last years, is the ability to develop and screen large antibody libraries for a specific target. This development is a direct consequence of phage display—a technology that enables the display of billions of proteins on top of the viral capsule. The phage display technology was followed by more technologies such as yeast display and ribosome display.

Previous antibody libraries were developed by amplifying human B cells or synthesizing a completely artificial library. Antibodies cloned from B cells may not represent the full diversity of the immune system and also may have a bias towards a certain clone of sequences. Synthetic libraries may produce immunogenic antibodies that can potentially trigger an immune response in patients.

Some libraries were constructed with human sequences. Although the sequences of these antibodies are human, they weren't optimized for stability or developability and may raise problems upon reaching the clinical setting. More such problems are recognized later in the process, the more costly it becomes.

Therapeutic antibodies must fulfill a high standard with regard to their developability, stability, immunogenicity, and functional activity. Previous generation antibody libraries, although large in number, couldn't accurately account for the vast majority of molecules in terms of stability and developability. These qualities were only determined once the antibody was screened and tested. Given that sorting methods (e.g. flow-cytometry or phage display) are known to be bound by approximately 10⁷ (flow cytometry) to 10¹¹ (phage display) variants, a reliable antibody library should be optimized in a way to maximize that every construct is developable and non-immunogenic, as well as be optimized for stability and binding specificity, to lower the probability of failure in later stages.

Most importantly, for an antibody to function as a drug, it often inhibits or facilitates an interaction between two protein members. For this inhibition or facilitation to occur, the antibody generally binds the target at the same space as the interacting partner and with better (or no worse) affinity.

This disclosure presents a pipeline in which a developable fully human antibody library that is directed towards specific epitope, is generated and optimized by computational tools.

Accordingly, there exists a need for an improved system and method for generating an antibody library.

### SUMMARY OF THE INVENTION

In one embodiment, the invention provides a computer implemented method for generating a library of antibodies, the method comprising: generating one or more seed structures based on one or more predetermined amino acid sequences of a complementarity determining region (CDR), one or more predetermined variable heavy (VH) and variable light (VL) structural framework (VH/VL) pairs, or a combination thereof; providing a predetermined epitope; docking said one or more seed structures on said epitope; evaluating one or more motifs of said one or more seed structures for one or more predetermined developability properties; and identifying one or more target structures in order to generate a library, thereby generating a library of antibodies.

In another embodiment, the invention provides a system for generating a library of antibodies, the system comprising: a seed structure generation unit that generates one or more seed structures based on one or more predetermined amino acid sequences of a complementarity determining region (CDR), one or more predetermined variable heavy (VH) and variable light (VL) structural framework (VH/VL) pairs, or a combination thereof; an epitope unit that provides a predetermined epitope; a docking unit that facilitates docking said one or more seed structures on said epitope; an evaluation unit that evaluates one or more motifs of said one or more seed structures for one or more predetermined developability properties; and a library generation unit that identifies one or more target structures in order to generate a library of antibodies.

In another embodiment, the invention provides a computer readable storage media comprising instructions to perform a method for generating a library of antibodies, the method comprising: generating one or more seed structures based on one or more predetermined amino acid sequences of a complementarity determining region (CDR), one or more predetermined variable heavy (VH) and variable light (VL) structural framework (VH/VL) pairs, or a combination thereof; providing a predetermined epitope; docking said one or more seed structures on said epitope; evaluating one or more motifs of said one or more seed structures for one or more predetermined developability properties; and identifying one or more target structures in order to generate a library, thereby generating a library of antibodies.

In another embodiment, the invention provides a computer implemented method for generating a library of antibodies, the method comprising: obtaining a first amino acid sequence of a complementarity determining region (CDR) associated with a heavy chain and a second amino acid sequence of a CDR associated with a light chain from a database of CDR sequences; obtaining one or more variable heavy (VH) and variable light (VL) structural framework (VH/VL) pairs, wherein each of said pair having one or more predetermined developability properties that facilitate for screening antibodies; analyzing said amino acid sequences and said VH/VL pairs with the use of a macro-molecular algorithmic unit to generate one or more seed structures; providing a predetermined epitope; docking said one or more seed structures on said epitope; evaluating the docked seed structures for a shape complementarity and an epitope overlap; selecting one or more seed structures having a value exceeding a predetermined threshold level, wherein said value is associated with a shape complementarity score, an epitope overlap score, or a combination thereof; evaluating one or more motifs of the selected structures to determine whether said one or more motifs exhibit a negative effect for one or more predetermined developability properties; and identifying one or more target structures based on the determination of said negative effect of said one or more motifs in order to generate a library, thereby generating a library of antibodies.

In another embodiment, the invention provides a system for generating a library of antibodies, the method comprising: a complementarity determining region (CDR) unit that facilitates obtaining a first amino acid sequence of a CDR associated with a heavy chain and a second amino acid sequence of a CDR associated with a light chain from a database of CDR sequences; a framework unit that facilitates obtaining one or more variable heavy (VH) and variable light (VL) structural framework (VH/VL) pairs, wherein each of said pair having one or more predetermined developability properties that facilitate for screening antibodies; an analysis unit that facilitates analyzing said amino acid sequences and said VH/VL pairs with the use of a macro-molecular algorithmic unit to generate one or more seed structures; an epitope unit that provides a predetermined epitope; a docking unit that facilitates docking said one or more seed structures on said epitope; an evaluation unit that facilitates evaluating the docked seed structures for a shape complementarity and an epitope overlap; a selection unit that facilitates selecting one or more seed structures having a value exceeding a predetermined threshold level, wherein said value is associated with a shape complementarity score, an epitope overlap score, or a combination thereof; a motif evaluation unit that facilitates evaluating one or more motifs of the selected structures to determine whether said one or more motifs exhibit a negative effect for one or more predetermined developability properties; and a library generation unit that facilitates identifying one or more target structures based on the determination of said negative effect of said one or more motifs in order to generate a library, thereby generating a library of antibodies.

In another embodiment, the invention provides a computer readable storage media comprising instructions to perform a method for generating a library of antibodies, the method comprising: obtaining a first amino acid sequence of a complementarity determining region (CDR) associated with a heavy chain and a second amino acid sequence of a CDR associated with a light chain from a database of CDR sequences; obtaining one or more variable heavy (VH) and variable light (VL) structural framework (VH/VL) pairs, wherein each of said pair having one or more predetermined developability properties that facilitate for screening antibodies; analyzing said amino acid sequences and said VH/VL pairs with the use of a macro-molecular algorithmic unit to generate one or more seed structures; providing a predetermined epitope; docking said one or more seed structures on said epitope; evaluating the docked seed structures for a shape complementarity and an epitope overlap; selecting one or more seed structures having a value exceeding a predetermined threshold level, wherein said value is associated with a shape complementarity score, an epitope overlap score, or a combination thereof; evaluating one or more motifs of the selected structures to determine whether said one or more motifs exhibit a negative effect for one or more predetermined developability properties; and identifying one or more target structures based on the determination of said negative effect of said one or more motifs in order to generate a library, thereby generating a library of antibodies.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements:

FIG. 1 illustrates a system for generating a library of antibodies, according to one embodiment of the invention.

FIG. 2 illustrates a flow chart of a method for generating a library of antibodies, according to one embodiment of the invention.

FIG. 3 illustrates a flow chart of a process of generating structural seeds for the docking step, using structure optimization (modeling) and sequence optimization (design), and PSSM to compute probabilities for amino acid preferences, according to one embodiment of the invention.

FIG. 4 illustrates a flow chart of a process of generating structural seeds for the docking step, using structure optimization, according to one embodiment of the invention.

FIG. 5 illustrates a flow chart of a process of calculating for each seed its best possible docking orientations with respect to the target in question and a predefined or pre-calculated epitope, according to one embodiment of the invention. These orientations can be served as starting structures for the design step.

FIG. 6 illustrates a flow chart of a process of calculating for each selected starting structure its optimized sequence, conformation and orientation with respect to the target, and the removal of motifs that may affect developability and/or immunogenicity, according to one embodiment of the invention.

FIG. 7 shows a germline configuration of an antibody molecule.

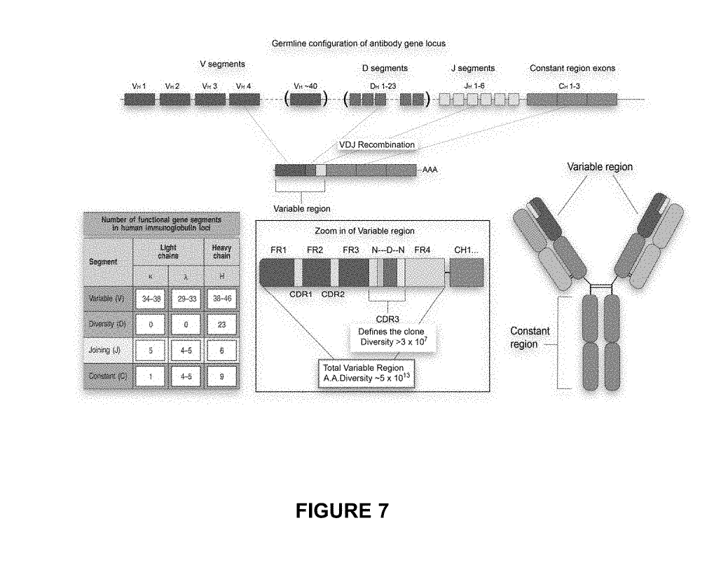

FIG. 8 shows a schematic drawing of an antibody molecule.

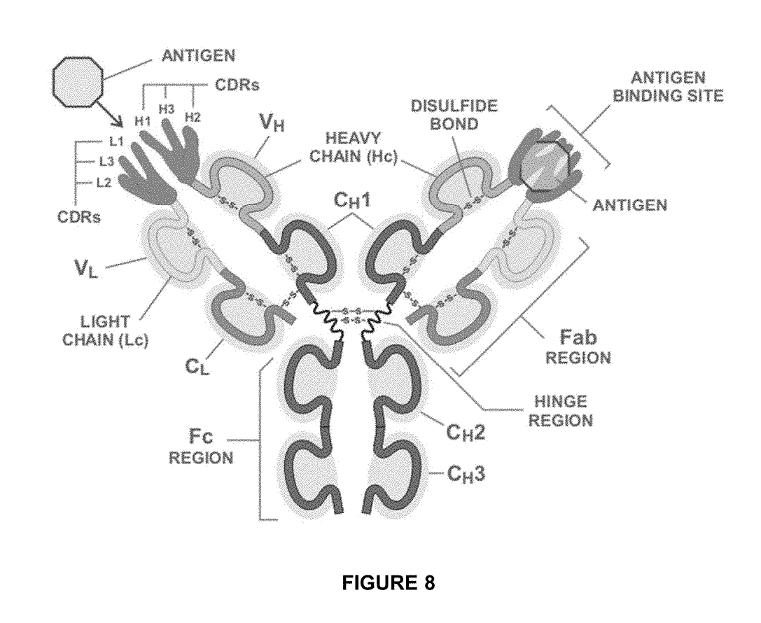

FIG. 9 shows the outputs Models of antibody (scFV)-ligand complexes together with the wild type ligand, demonstrating the overlap in binding site.

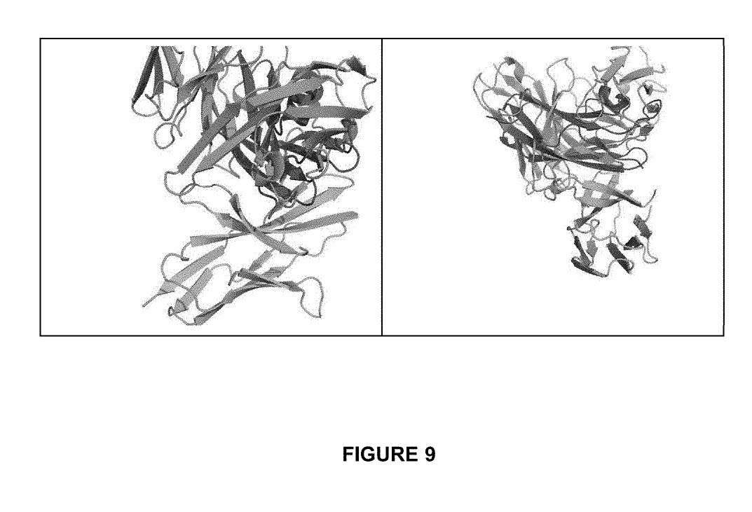

### DETAILED DESCRIPTION OF THE INVENTION

The invention provides system and method for generating an antibody library. Specifically, the invention relates to a computer-implemented system and method for generating a library of antibodies based on a predetermined epitope.

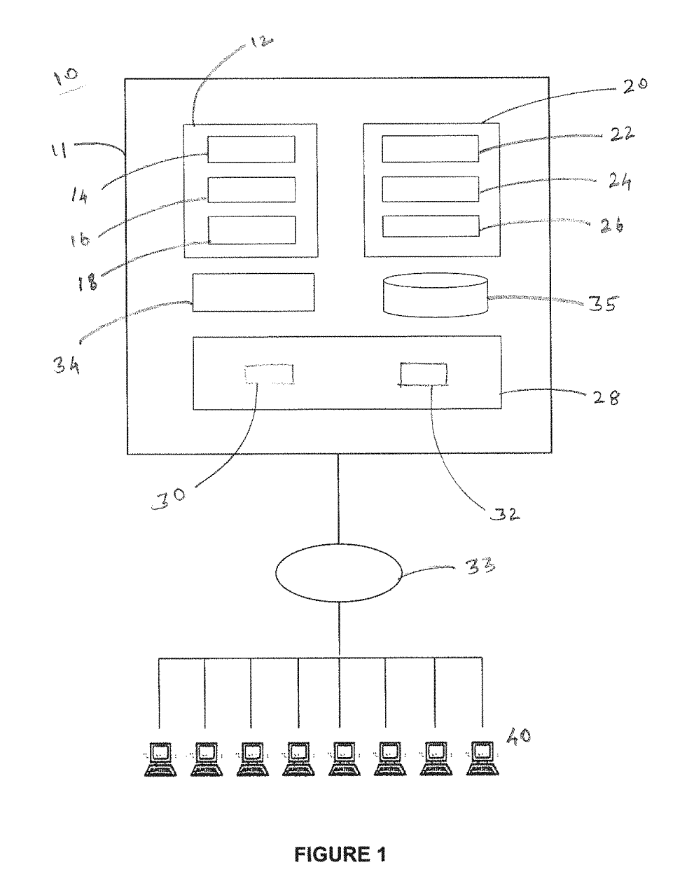

FIG. 1 schematically illustrates one arrangement of a system for generating an antibody library. Although the FIG. 1 environment shows an exemplary conventional general-purpose digital environment, it will be understood that other computing environments may also be used. For example, one or more embodiments of the present invention may use an environment having fewer than or otherwise more than all of the various aspects shown in FIG. 1, and these aspects may appear in various combinations and sub-combinations that will be apparent to one of ordinary skill in the art.

As shown in FIG. 1, a user computer 10 can operate in a networked environment using logical connections to one or more remote computers, such as a remote server 11. The server 11 can be a web server, a router, a network PC, a peer device or other common network node, and typically includes many or all of the elements of a computer. It will be appreciated that the network connections shown in FIG. 1 are exemplary and other techniques for establishing a communications link between the computers can be used. The connection may include a local area network (LAN) and a wide area network (WAN). The existence of any of various well-known protocols such as TCP/IP, Ethernet, FTP, HTTP and the like is presumed, and the system can be operated in a client-server configuration to permit a user to retrieve web pages from a web-based server. Any of various conventional web browsers as well as non-web interfaces can be used to display and manipulate data.

In one aspect, an antibody library can be generated in an online environment. As illustrated in FIG. 1, a user (e.g., researcher) 41 has a user computer 40 with Internet access that is operatively coupled to server 11 via a network 33, which can be an internet or intranet. User computer 40 and server 11 implement various aspects of the invention that is apparent in the detailed description. For example, user computer 40 may be in the form of a personal computer, a tablet personal computer or a personal digital assistant (PDA). Tablet PCs interprets marks made using a stylus in order to manipulate data, enter text, and execute conventional computer application tasks such as spreadsheets, word processing programs, and the like. User computer 40 is configured with an application program that communicates with server 11. This application program can include a conventional browser or browser-like programs.

In one embodiment, server 11 may include a plurality of programmed platforms or units, for example, but are not limited to, a seed generation platform 12, docking platform 20, design platform 28, and an epitope unit 34. Seed generation platform 12 may include one or more programmable units, for example, but are not limited to, a complementarity determining region (CDR) unit 14, a framework unit 16, and an analysis unit 18. Docking platform 20 may include a plurality of programmed platforms or units, for example, but are not limited to, a docking unit 22, an evaluation unit 24, and a selection unit 26. Design platform 28 may include a plurality of programmed platforms or units, for example, but are not limited to, a motif evaluation unit 30 and a library generation unit 32.

The term “platform” or “unit,” as used herein, may refer to a collection of programmed computer software codes for performing one or more tasks.

CDR 14 unit may facilitate a user to obtain a first amino acid sequence of a CDR associated with a heavy chain and a second amino acid sequence of a CDR associated with a light chain from a database 35 of CDR sequences. In one embodiment, the first amino acid sequence is H3 sequence of CDR3. In another embodiment, the first amino acid sequence is L3 sequence of CDR3. In one example database 35 is a CDR3 sequence database.

Framework unit 16 may facilitate a user to obtain one or more variable heavy (VH) and variable light (VL) structural framework (VH/VL) pairs. Each of the pair may have one or more predetermined developability properties that facilitate for screening antibodies. The predetermined developability properties may also facilitate for selecting one or more desirable VH/VL pairs. Examples of a predetermined developability property include, for example, but not limited to, an expression rate (mg/L), a relative display rate, a thermal stability (Tₘ), an aggregation propensity, a serum half-life, an immunogenicity, and a viscosity. In a particular embodiment, the predetermined developability property is an immunogenicity.

Analysis unit 18 may facilitate for analyzing the amino acid sequences and the VH/VL pairs with the use of a macro-molecular algorithmic unit to generate one or more seed structures.

The macro-molecular algorithmic unit may facilitate for evaluating the amino acid sequence of H3 loop, L3 loop, or a combination thereof. The macro-molecular algorithmic unit can be used to modify or optimize the amino acid sequence of H3 loop, L3 loop, or a combination thereof. In one embodiment, the amino acid sequence of H3 loop, L3 loop, or a combination thereof can be modified or optimized based on a Point Specific Scoring Matrix (PSSM). In another embodiment, the amino acid sequence of H3 loop, L3 loop, or a combination thereof can be modified or optimized based on one or more VH/VL pairs.

In one aspect, one or more seed structures are generated based on an energy function of H3 loop, L3 loop, VH/VL pair or a combination thereof. In another aspect, one or more seed structures are generated based on humanization of the structures.

Epitope unit 34 may facilitate for providing a predetermined epitope. In one example, the epitope is determined based on a subset of a protein. In another example, the epitope has one or more residues that interact with its interacting partner at a predetermined distance. In one embodiment, the distance is <4 A. Other suitable distances are also encompassed within the scope of the invention.

Docking unit 22 may facilitate for docking one or more seed structures on the epitope. Evaluation unit 24 may facilitate for evaluating the docked seed structures for a shape complementarity and an epitope overlap.

Selection unit 26 may facilitate for selecting one or more seed structures having a value exceeding a predetermined threshold level. In one embodiment, the predetermined threshold level is based on a shape complementarity score. In another embodiment, the predetermined threshold level is based on an epitope overlap score. In some embodiments, the predetermined threshold level is based a combination of a shape complementarity score and an epitope overlap score.

In some embodiments, one or more selected seed structures can be optimized using a simulated annealing process which is an adaptation of the Monte Carlo method to generate sample states of a thermodynamic system. In another embodiment, the simulated annealing process is composed of rigid body minimization, antibody H3-L3 sequence optimization, optimizing the packing of interface and core, optimizing the backbone of antibody, optimizing the light and heavy chain orientation, optimizing the antibody as monomer, or a combination thereof.

Motif evaluation unit 30 may facilitate for evaluating one or more motifs of the selected structures to determine whether one or more motifs exhibit a negative effect for one or more predetermined developability properties. In some embodiments, the one or more motifs with negative effects are removed. In a particular embodiment, an immunogenic motif is removed.

In one embodiment, CDR regions are mutated according to a Point Specific Scoring Matrix (PSSM) and the evaluation may be performed by evaluating an energy score that is derived from the algorithmic unit.

Library generation unit 32 may facilitate for identifying one or more target structures based on the determination of any negative effect of one or more motifs in order to generate a library.

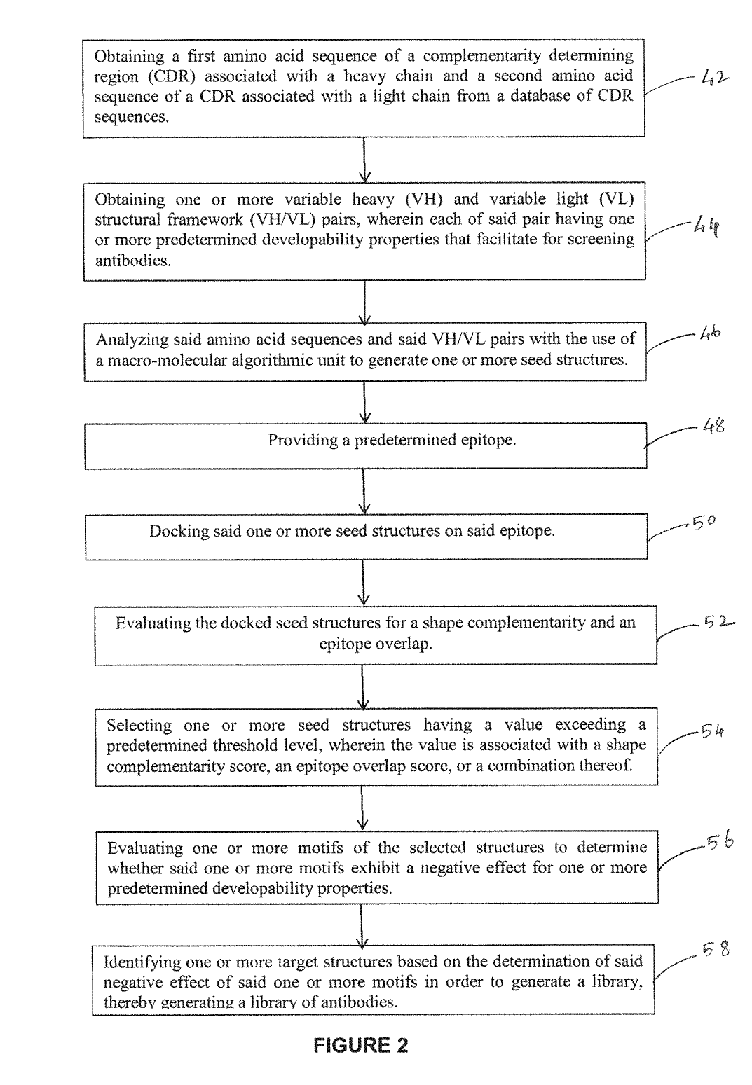

FIG. 2 illustrates a method for generating a library of antibodies, according to one embodiment of the invention. As shown in item 42, a first amino acid sequence of a CDR associated with a heavy chain and a second amino acid sequence of a CDR associated with a light chain can be obtained from database 35 of CDR sequences. As shown in item 44, one or more variable heavy (VH) and variable light (VL) structural framework (VH/VL) pairs can be obtained. Each of the pair may have one or more predetermined developability properties that facilitate for screening antibodies. As shown in item 46, the amino acid sequences and the VH/VL pairs can be analyzed with the use of a macro-molecular algorithmic unit to generate one or more seed structures. As shown in item 48, a predetermined epitope can be provided. As shown in item 50, one or more seed structures can be docked on the epitope. As shown in item 52, the docked seed structures can be evaluated for a shape complementarity, an epitope overlap, or a combination thereof. As shown in item 54, one or more seed structures having a value passing or exceeding a predetermined threshold level can be selected. The value and the predetermined threshold level may be associated with a shape complementarity score, an epitope overlap score, or a combination thereof. As shown in item 56, evaluating one or more motifs of the selected structures can be evaluated to determine whether one or more motifs exhibit a negative effect for one or more predetermined developability properties. As shown in item 58, one or more target structures can be identified based on the determination of said negative effect of said one or more motifs in order to generate a library.

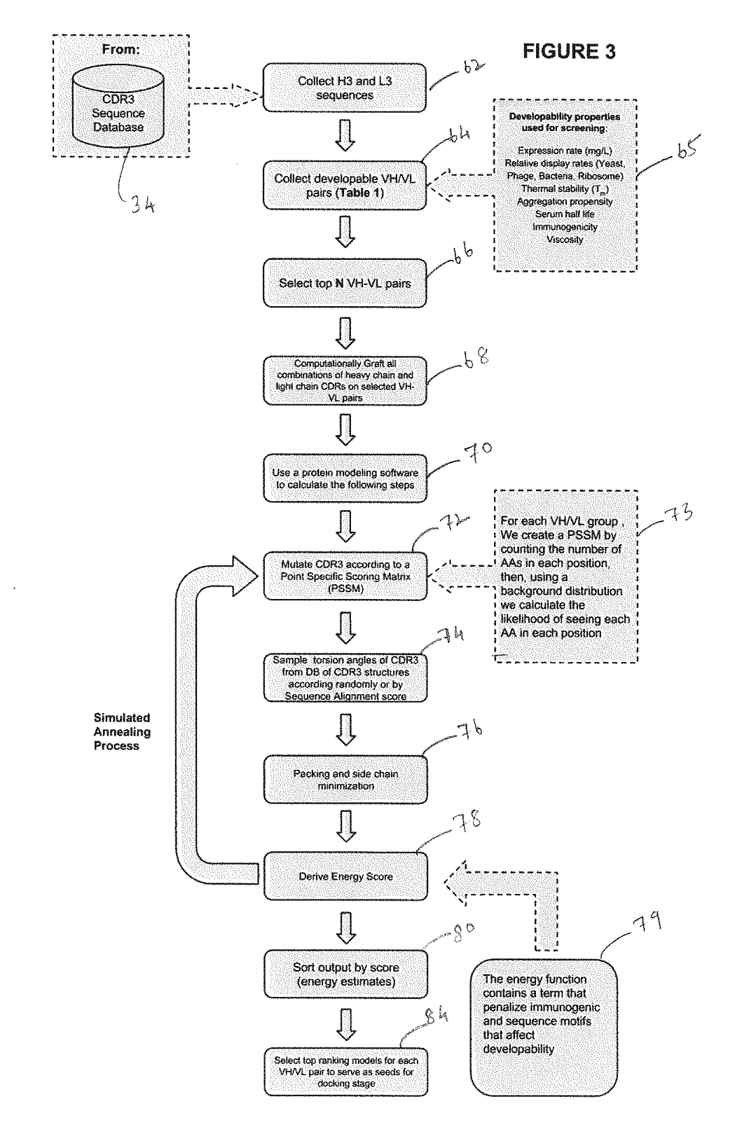

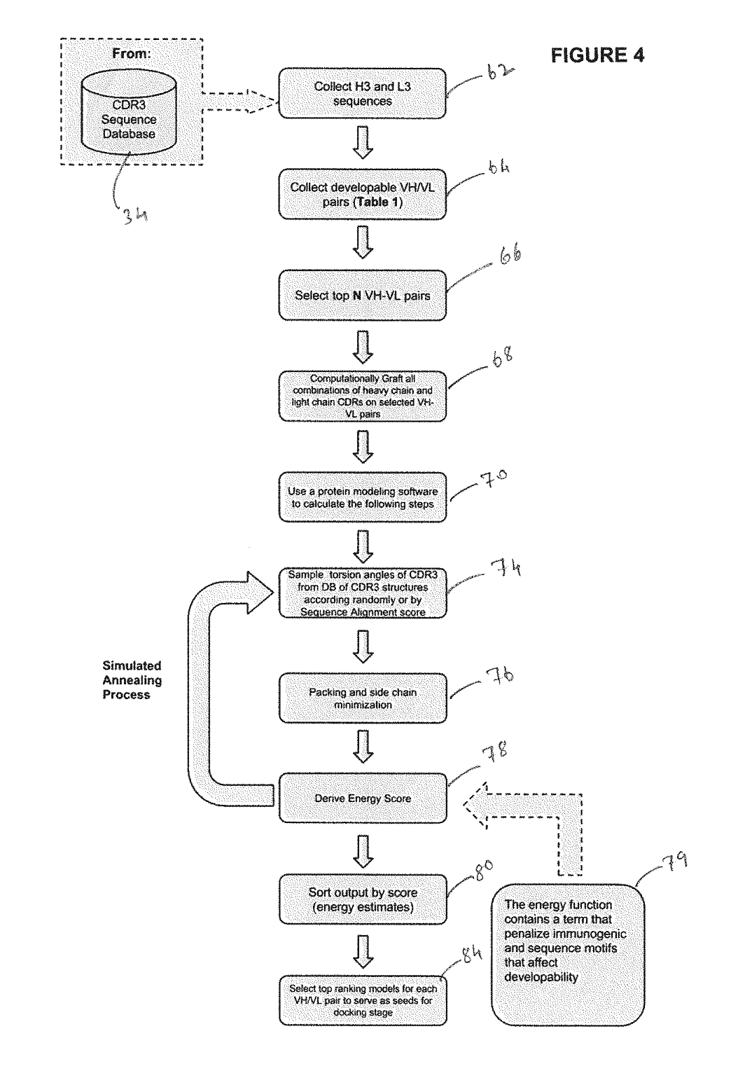

FIG. 3 shows a process of generating structural seeds for the docking step, using structure optimization (modeling) and sequence optimization (design) possibly approach PSSM to compute probabilities for amino acid preferences, according to one embodiment of the invention. As shown in item 62, H3 and L3 sequences can be collected from CDR sequence database 35. As shown in item 64, one or more VL/VH pairs having one or more predetermined developability properties can be collected. As shown in item 66, the collected VL/VH pairs can be evaluated to select top VL/VH pairs, for example, VL/VH pairs having the best developability properties. As shown in item 68, one or more combinations of heavy chain and light chain CDRs can be computationally grafted on the selected VL/VH pairs. As shown in item 70, a protein modeling software can be used to calculate one or more scores. As shown in item 72, CDR3 can be mutated according to a Point Specific Scoring Matrix (PSSM). In one example, PSSM can be created by counting the number of amino acids, and then the likelihood of each amino acid in each position can be calculated using a background distribution. As shown in item 74, torsion angles of CDR3 from a database of CDR3 structures can be sampled randomly or according to a sequence alignment score. In some embodiments, as shown in FIG. 4, without the step of mutating CDR3 according to PSSM, torsion angles of CDR3 from a database of CDR3 structures can be sampled randomly or according to a sequence alignment score.

As shown in item 76, a packing and a side chain minimization can be performed. As shown in item 78, an energy score can be derived. As shown in item 79, immunogenic or sequence motif affecting developability can be penalized to determine the energy function. As shown in item 80, an output score can be sorted based on energy estimates. As shown in item 84, one or more top ranking structures or models can be selected for each VH/VL pair to serve as seeds for docking stage.

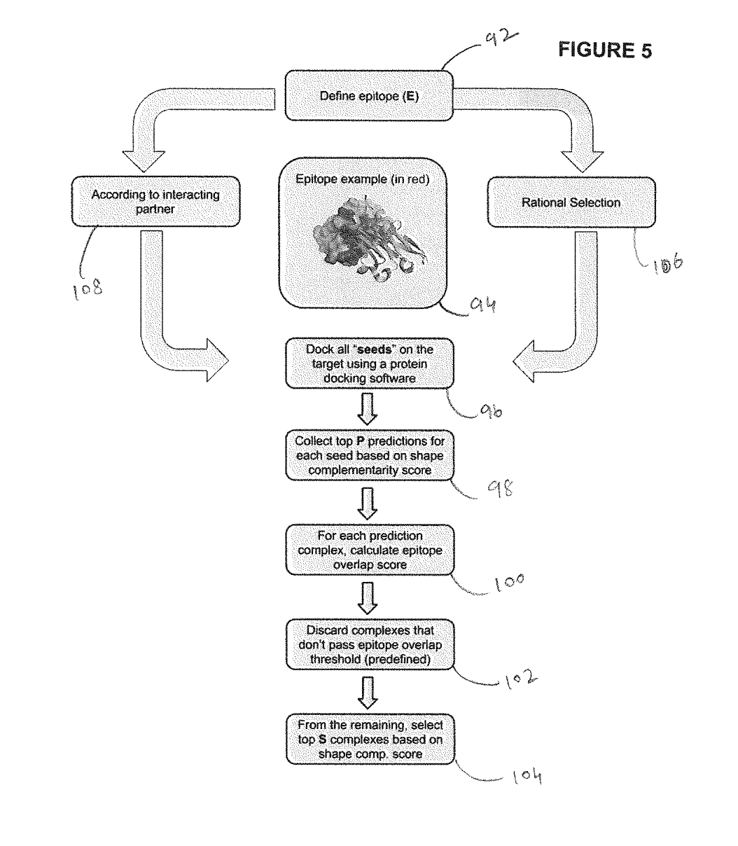

FIG. 5 shows a process of calculating for each seed its best possible docking orientations with respect to the target in question and a predefined or pre-calculated epitope, according to one embodiment of the invention. As shown in item 92, an epitope can be defined. Item 94 shows an example of an epitope. In one embodiment, as shown in item 108, an epitope can be defined according to an interacting partner. In another embodiment, as shown in item 106, an epitope can be defined based on rational selection. As shown in item 96, the seeds can be docked on target epitope using a protein docking software. As shown in item 98, based on a shape complementarity score, one or more top seed structures can be collected. As shown in item 100, an epitope overlap score can be calculated. As shown in item 102, one or more complexes or structures that do not pass epitope overlap threshold level can be discarded. As shown in item 104, one or more complexes or structures can be selected based on a shape complementarity score.

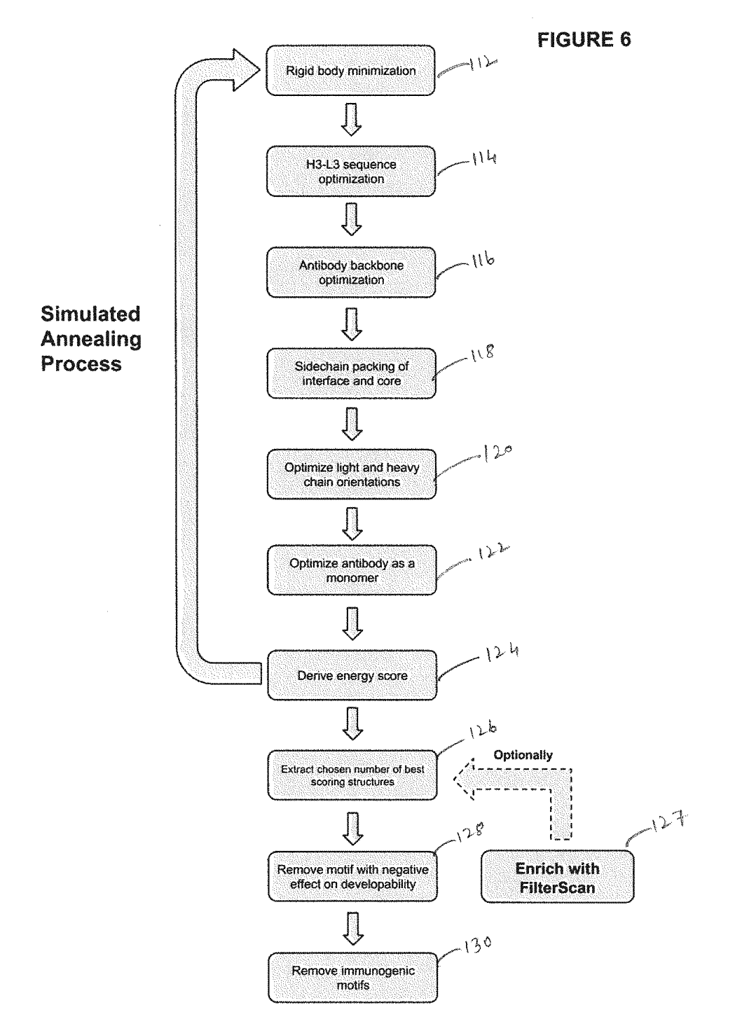

FIG. 6 shows a process of calculating for each selected starting structure its optimized sequence, conformation and orientation with respect to the target, and the removal of motifs that may affect developability and/or immunogenicity, according to one embodiment of the invention. As shown in FIG. 6, a simulated annealing process can be performed based on, for example, rigid body minimization (112), H3-L3 sequence optimization (114), antibody backbone optimization (116), sidechain packing of interface and core (118), optimization of light and heavy chain orientations (120), and optimization of antibody as a monomer (122). As shown in item 124, an energy score can be derived. As shown in item 126, best scoring structures can be extracted. In some embodiments, as shown in item 127, filtration can be performed for further enrichment. As shown in items 128 and 130, one or more motifs with negative effects on developability or one or more immunogenic motifs can be removed. As a result, an antibody library can be generated.

The following examples are presented in order to more fully illustrate the preferred embodiments of the invention. They should in no way be construed, however, as limiting the broad scope of the invention.

### EXAMPLES

### Example 1

Our invention utilizes computational processing power to compute optimal antibody molecules that bind a predefined epitope of a selected target polypeptide molecule. Given a computer system and a macro molecular modeling software that is able to approximate the free energy of a protein molecule (a.k.a free energy score, and/or score may be used interchangeably) the algorithm is detailed below and is divided to 3 sections:
1. Seed generation
2. Docking
3. Design

Each of the 2 first sections generates the input for the next section. Unless otherwise stated, all procedures described here (such as grafting, mutating) are purely computational.

### Stage 1: Seed Generation

1. Collect H3+L3 sequences from a data set (either human or other organism):
   a. B cell repertoire
   b. existing PDB structures
2. Collect VH/VL pairs of antibody frameworks that have good developability properties (F) (See Table 1)
3. Use a macro-molecular modeling software to either:
   a. model (do not change amino acid sequence of H3+L3 loops)
   b. design (optimize the amino acid sequence of the loops according to PSSM and VH/VL structure)
the H3-L3 combinations on top of all VH/VL pairs of antibody frameworks
4. Select top N best energy scoring structures (VH-H3-VL-L3) for each framework (NxF) to serve as seeds
5. If started from non-human framework, humanize at the end.
Stage 2: Docking
6. Define epitope (E) (E—set of protein residues)
   a. Rational selection—manually define a subset of protein residues to serve as epitope.
   b. According to interacting partner—define the epitope as the set of all residues that “interact” (distance to partner <4 A) with that target's interacting partner.
7. dock all seeds using a protein docking software on target
8. Collect top P best predictions complexes for each seed, based on shape complementarity score
9. for each complex P calculate epitope overlap.

### Example

a. Calculate Eₚ—the set of residues that “interact” (distance to partner <4 A) with the target's interacting partner
b. Calculate:

$$\frac{E ⋂ {E}_{p}}{E ⋃ {E}_{p}}$$

for each complex
Another possibility—calculate just the overlap for the CDRs.
10. Discard all complexes that don't pass a predefined epitope overlap threshold
11. From the complexes that pass the threshold, select the S complexes that have the best shape complementarity score (according to the docking software)

### Stage 3: Design

1. Use a protein modeling software and a predefined energy function to iterate the following as a Monte Carlo with Simulated Annealing process:
   a. Rigid body minimization
   b. Antibody H3-L3 sequence optimization
   c. optimize packing of interface and core
   d. optimize backbone of antibody
   e. optimize light and heavy chain orientation
   f. optimize antibody as monomer
2. Extract a chosen number of best scoring structures
3. Optionally, Enrich the set of selected antibodies by running FilterScan:
   a. Go over each position in the H3 and L3 loops and try all possible mutations or mutations according to PSSM and a probability threshold (mutations that are more common according to the PSSM will have a higher probability of being sampled)
   b. Evaluate energy score and accept only if improved.
4. For each chosen structure:
   a. Remove motifs that may have negative effect on developability
   b. Remove immunogenic motifs.

TABLE 1
Developability properties used for selecting VH/VL frameworks
Developability properties used for screening

Expression rate (mg/L)
Relative display rates (Yeast, Phage, Bacteria, Ribosome)
Thermal stability (Tₘ)
Aggregation propensity
Serum half life
Immunogenicity
Viscosity

### Implementation

On an amazon cloud, installed with a protein modeling software:
1. Start with 50,000 antibody models, dock each of them on target.
2. Calculate overlap with interaction site of the ligand (epitope) take the best 10% of the models
3. Run a design algorithm on each of the 10%, generate 5 designs for each. (On our cluster, it took 2 hours for a single CPU to generate 1 design. Overall, 50,000 CPU hours)
4. Amplify the variability of the designs by running the FilterScan algorithm.
5. Pick the best scoring 50,000 for synthesis.

Alternatively, one can start with more antibody models in the first step, and omit the filterscan step. Starting from a larger number of antibody models should yield a library with a larger diversity, as the filterscan algorithm generates just one mutation per model. Starting from a larger number of antibody models however, requires more CPU hours and therefore is more costly.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A computer implemented method for generating a library of antibodies, the method comprising:
generating one or more seed structures based on one or more predetermined amino acid sequences of a complementarity determining region (CDR), one or more predetermined variable heavy (VH) and variable light (VL) structural framework (VH/VL) pairs, or a combination thereof;
providing a predetermined epitope;
docking said one or more seed structures on said epitope;
evaluating one or more motifs of said one or more seed structures for one or more predetermined developability properties; and
identifying one or more target structures in order to generate a library, thereby generating a library of antibodies.

2. The method of claim 1, wherein the step of generating one or more seed structures comprising:
obtaining a first amino acid sequence of a complementarity determining region (CDR) associated with a heavy chain and a second amino acid sequence of a CDR associated with a light chain from a database of CDR sequences;
obtaining one or more variable heavy (VH) and variable light (VL) structural framework (VH/VL) pairs, wherein each of said pair having one or more predetermined developability properties that facilitate for screening antibodies; and
analyzing said amino acid sequences and said VH/VL pairs with the use of a macro-molecular algorithmic unit to generate one or more seed structures.

3. The method of claim 1, further comprising:
evaluating the docked seed structures for a shape complementarity and an epitope overlap;
selecting one or more seed structures having a value exceeding a predetermined threshold level, wherein said value is associated with a shape complementarity score, an epitope overlap score, or a combination thereof.

4. The method of claim 1, wherein the step of evaluating one or more motifs comprising evaluating one or more motifs of the selected structures to determine whether said one or more motifs exhibit a negative effect for one or more predetermined developability properties.

5. The method of claim 1, wherein the step of identifying one or more target structures is based on the determination of presence or absence of said negative effect of said one or more motifs.

6. The method of claim 2, wherein said first amino acid sequence is H3 sequence of CDR3.

7. The method of claim 2, wherein said first amino acid sequence is L3 sequence of CDR3.

8. The method of claim 2, wherein said database is a CDR3 sequence database.

9. The method of claim 2, wherein said one or more predetermined developability properties facilitate for selecting one or more VH/VL pairs.

10. The method of claim 2, wherein at least one of said one or more predetermined developability properties is an immunogenicity.

11. The method of claim 2, wherein at least one of said one or more predetermined developability properties is an expression rate (mg/L), a relative display rate, a thermal stability (Tₘ), an aggregation propensity, a serum half-life, an immunogenicity, or a viscosity.

12. The method of claim 2, wherein said macro-molecular algorithmic unit evaluates the amino acid sequence of H3 loop, L3 loop, or a combination thereof.

13. The method of claim 2, wherein said macro-molecular algorithmic unit modifies or optimizes the amino acid sequence of H3 loop, L3 loop, or a combination thereof, based on a Point Specific Scoring Matrix (PSSM) and said one or more VH/VL pairs.

14. The method of claim 2, wherein said one or more seed structures are generated based on an energy function of H3 loop, L3 loop, said one or more VH/VL pairs or a combination thereof.

15. The method of claim 2, wherein said one or more seed structures are generated based on humanization of said structures.

16. The method of claim 1, wherein said predetermined epitope is a subset of a protein.

17. The method of claim 1, wherein said predetermined epitope has one or more residues that interact with its interacting partner at a distance <4 A.

18. The method of claim 3, further comprising evaluating the selected seed structures for a simulated annealing process.

19. The method of claim 18, wherein said annealing process is performed by a Monte Carlo simulation.

20. The method of claim 18, wherein said annealing process is performed based on rigid body minimization, antibody H3-L3 sequence optimization, optimizing the packing of interface and core, optimizing the backbone of antibody, optimizing the light and heavy chain orientation, optimizing the antibody as monomer, or a combination thereof.

21. The method of claim 4, wherein the step of evaluation optionally comprising analyzing one or more residues in the H3 or L3 loops to determine a mutation based on a Point Specific Scoring Matrix (PSSM) or a probability threshold and evaluate an energy score.

22. The method of claim 4, wherein the step of evaluation comprising removing immunogenic motifs.

23. The method of claim 4, wherein the step of evaluation comprising removing one or more motifs with negative effects on one or more predetermined developability properties.

24. A system for generating a library of antibodies, the system comprising:
a seed structure generation unit that generates one or more seed structures based on one or more predetermined amino acid sequences of a complementarity determining region (CDR), one or more predetermined variable heavy (VH) and variable light (VL) structural framework (VH/VL) pairs, or a combination thereof;
an epitope unit that provides a predetermined epitope;
a docking unit that facilitates docking said one or more seed structures on said epitope;
an evaluation unit that evaluates one or more motifs of said one or more seed structures for one or more predetermined developability properties; and
a library generation unit that identifies one or more target structures in order to generate a library of antibodies.

25. A computer readable storage media comprising instructions to perform a method for generating a library of antibodies, the method comprising:
generating one or more seed structures based on one or more predetermined amino acid sequences of a complementarity determining region (CDR), one or more predetermined variable heavy (VH) and variable light (VL) structural framework (VH/VL) pairs, or a combination thereof;
providing a predetermined epitope;
docking said one or more seed structures on said epitope;
evaluating one or more motifs of said one or more seed structures for one or more predetermined developability properties; and
identifying one or more target structures in order to generate a library, thereby generating a library of antibodies.

26. A computer implemented method for generating a library of antibodies, the method comprising:
obtaining a first amino acid sequence of a complementarity determining region (CDR) associated with a heavy chain and a second amino acid sequence of a CDR associated with a light chain from a database of CDR sequences;
obtaining one or more variable heavy (VH) and variable light (VL) structural framework (VH/VL) pairs, wherein each of said pair having one or more predetermined developability properties that facilitate for screening antibodies;
analyzing said amino acid sequences and said VH/VL pairs with the use of a macro-molecular algorithmic unit to generate one or more seed structures;
providing a predetermined epitope;
docking said one or more seed structures on said epitope;
evaluating the docked seed structures for a shape complementarity and an epitope overlap;
selecting one or more seed structures having a value exceeding a predetermined threshold level, wherein said value is associated with a shape complementarity score, an epitope overlap score, or a combination thereof;
evaluating one or more motifs of the selected structures to determine whether said one or more motifs exhibit a negative effect for one or more predetermined developability properties; and
identifying one or more target structures based on the determination of said negative effect of said one or more motifs in order to generate a library, thereby generating a library of antibodies.

27. A system for generating a library of antibodies, the method comprising:
a complementarity determining region (CDR) unit that facilitates obtaining a first amino acid sequence of a CDR associated with a heavy chain and a second amino acid sequence of a CDR associated with a light chain from a database of CDR sequences;
a framework unit that facilitates obtaining one or more variable heavy (VH) and variable light (VL) structural framework (VH/VL) pairs, wherein each of said pair having one or more predetermined developability properties that facilitate for screening antibodies;
an analysis unit that facilitates analyzing said amino acid sequences and said VH/VL pairs with the use of a macro-molecular algorithmic unit to generate one or more seed structures;
an epitope unit that provides a predetermined epitope;
a docking unit that facilitates docking said one or more seed structures on said epitope;
an evaluation unit that facilitates evaluating the docked seed structures for a shape complementarity and an epitope overlap;
a selection unit that facilitates selecting one or more seed structures having a value exceeding a predetermined threshold level, wherein said value is associated with a shape complementarity score, an epitope overlap score, or a combination thereof;
a motif evaluation unit that facilitates evaluating one or more motifs of the selected structures to determine whether said one or more motifs exhibit a negative effect for one or more predetermined developability properties; and
a library generation unit that facilitates identifying one or more target structures based on the determination of said negative effect of said one or more motifs in order to generate a library, thereby generating a library of antibodies.

28. A computer readable storage media comprising instructions to perform a method for generating a library of antibodies, the method comprising:
obtaining a first amino acid sequence of a complementarity determining region (CDR) associated with a heavy chain and a second amino acid sequence of a CDR associated with a light chain from a database of CDR sequences;
obtaining one or more variable heavy (VH) and variable light (VL) structural framework (VH/VL) pairs, wherein each of said pair having one or more predetermined developability properties that facilitate for screening antibodies;
analyzing said amino acid sequences and said VH/VL pairs with the use of a macro-molecular algorithmic unit to generate one or more seed structures;
providing a predetermined epitope;
docking said one or more seed structures on said epitope;
evaluating the docked seed structures for a shape complementarity and an epitope overlap;
selecting one or more seed structures having a value exceeding a predetermined threshold level, wherein said value is associated with a shape complementarity score, an epitope overlap score, or a combination thereof;
evaluating one or more motifs of the selected structures to determine whether said one or more motifs exhibit a negative effect for one or more predetermined developability properties; and
identifying one or more target structures based on the determination of said negative effect of said one or more motifs in order to generate a library, thereby generating a library of antibodies.
